(19) United States
(12) Patent Application Publication
(10) Pub. No.: US 2010/0249720 A1
(43) Pub. Date: Sep. 30, 2010

(54) INTEGRATED DEVICE FOR THE STORAGE AND DELIVERY OF A BONE GRAFT OR OTHER IMPLANTABLE MATERIAL TO A SURGICAL SITE

(76) Inventors: Ashok Biyani, Sylvania, OH (US); Brian Blessinger, Holland, OH (US)

Correspondence Address: MACMILLAN SOBANSKI & TODD, LLC, ONE MARITIME PLAZA FIFTH FLOOR, 720 WATER STREET, TOLEDO, OH, 43604-1619, US

(21) Appl. No.: 12/751,129

(22) Filed: Mar. 31, 2010

Related U.S. Application Data
(60) Provisional application No. 61/164,926, filed on Mar. 31, 2009.

(51) Int. Cl. A61M 5/31; A61M 5/315; A61B 17/56

(52) U.S. Cl. 604/218; 604/187

(57) ABSTRACT

A device for the storage and delivery of a bone graft or other implantable material to a desired surgical site includes a storage portion that extends from a first end to a second end. The first end of the storage portion is adapted to have a bone graft or other implantable material transferred therethrough into the storage portion. The device also includes a delivery portion that extends from a first end to a second end. The first end of the delivery portion communicates with the second end of the storage portion to permit the movement of the bone graft or other implantable material therethrough. The second end of the delivery portion is adapted to be positioned at a desired surgical site. The device further includes a delivery mechanism that is provided within the storage portion and the delivery portion and is adapted to move the bone graft material from the storage portion through the delivery portion to a desired surgical site.

### STATEMENTS REGARDING FEDERALLY SPONSORED RESEARCH OR DEVELOPMENT AND CROSS REFERENCE TO RELATED APPLICATIONS

This invention was not made with any government support. This application claims the benefit of U.S. Provisional Application No. 61/164,926 filed Mar. 30, 2009, the disclosure of which is incorporated herein by reference.

### BACKGROUND OF THE INVENTION

This invention relates in general to the field of orthopedic surgery and, more specifically, to the area of spinal fusion surgery. In particular, this invention relates to an integrated device for the storage and delivery of a bone graft or other implantable material to a desired surgical site.

Spinal fusion surgery usually involves the placement of osteoinductive or osteoconductive materials in a desired surgical site to be fused. The materials used for the bone graph can originate from a variety of sources, including from the patient (autograft material), from other humans (allograft material), from synthetic sources, from non-human sources such as animals (xenograft material), and from naturally occurring minerals (alloplastic material). As used herein, the term “bone graft material” relates to these and any other similar material that can be used for this purpose.

In some instances, the process of implanting the bone graft material at a desired surgical site can be problematic. During a surgical procedure, the bone graft material may need to be transferred from a first container, in which the bone graft material is stored prior to use, to a second container, which typically is formed as a portion of a device for delivering the bone graft material to the surgical site. The transfer of the bone graft material from the first container to the second container is inconvenient at best and can result in undesirable splashing or spilling of the bone graft material. After the bone graft material has been transferred from the first container to the second container, known delivery devices are not well suited for delivering a metered or otherwise precise amount of the bone graft material deep at the surgical site in a well controlled manner. Thus, it would be desirable to provide an improved device that eliminates the need for transferring the bone graft material from the first container to the second container and that facilitates a metered or otherwise precise amount of bone graft material to be delivered to the surgical site.

### SUMMARY OF THE INVENTION

This invention relates to an integrated device for the storage and delivery of a bone graft or other implantable material to a desired surgical site. The device includes a storage portion that extends from a first end to a second end. The first end of the storage portion is adapted to have a bone graft or other implantable material transferred therethrough into the storage portion. The device also includes a delivery portion that extends from a first end to a second end. The first end of the delivery portion communicates with the second end of the storage portion to permit the movement of the bone graft or other implantable material therethrough. The second end of the delivery portion is adapted to be positioned at a desired surgical site. The device further includes a delivery mechanism that is provided within the storage portion and the delivery portion and is adapted to move the bone graft material from the storage portion through the delivery portion to a desired surgical site.

Various objects and advantages will become apparent to those skilled in the art from the following detailed description of the preferred embodiments, when read in light of the accompanying drawings.

### BRIEF DESCRIPTION OF THE DRAWINGS

FIG. 1 is a schematic side elevational view of three vertebrae of a portion of a human spine, an undamaged intervertebral disc extending between the first and second vertebrae, and a damaged intervertebral disc extending between the second and third vertebrae.

FIG. 2 is a schematic side elevational view similar to FIG. 1 wherein a portion of the damaged intervertebral disc has been removed, and wherein a portion of an integrated device for the storage and delivery of a bone graft or other implantable material in accordance with this invention is shown depositing the bone graft material within the damaged intervertebral disc.

FIG. 3 is an enlarged sectional elevational view of a portion of a first embodiment of the integrated storage and delivery device illustrated in FIG. 2.

FIG. 4 is a top plan view of the first embodiment of the integrated storage and delivery device illustrated in FIG. 3.

FIG. 5 is a sectional elevational view of a portion of a second embodiment of the integrated storage and delivery device illustrated in FIG. 2.

FIG. 6 is a sectional elevational view of a portion of a third embodiment of the integrated storage and delivery device illustrated in FIG. 2.

FIG. 7 is a sectional elevational view of a portion of a fourth embodiment of the integrated storage and delivery device illustrated in FIG. 2.

FIG. 8 is a top plan view of the fourth embodiment of the integrated storage and delivery device illustrated in FIG. 7.

FIG. 9 is a sectional elevational view of a fifth embodiment of the integrated storage and delivery device illustrated in FIG. 2.

FIG. 10 is a sectional elevational view of a modified version of the fifth embodiment of the integrated storage and delivery device illustrated in FIG. 9.

FIG. 11 is a sectional elevational view of a sixth embodiment of the integrated storage and delivery device illustrated in FIG. 2.

FIG. 12 is a sectional elevational view of a seventh embodiment of the integrated storage and delivery device illustrated in FIG. 2.

FIG. 13 is a sectional elevational view of an eighth embodiment of the integrated storage and delivery device illustrated in FIG. 2.

### DETAILED DESCRIPTION OF THE PREFERRED EMBODIMENTS

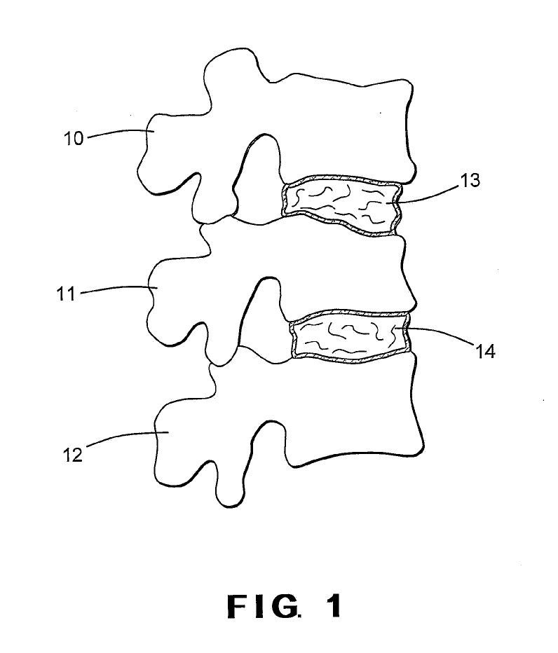

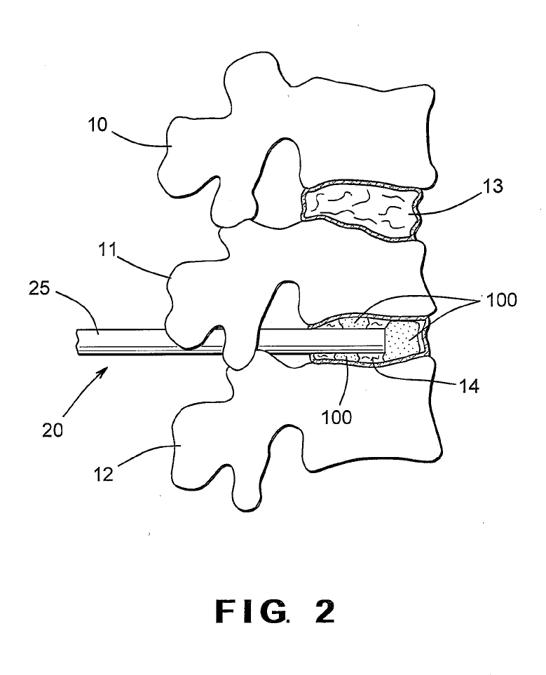

Referring now to the drawings, there is illustrated in FIGS. 1 and 2 three vertebrae 10, 11, and 12 of a portion of a human spine. The illustrated vertebrae 10, 11, and 12 may be located in any region of the human spine, but typically are located in the thoracic or lumbar regions. As shown in FIG. 1, the first and second vertebrae 10 and 11 are separated by a first fibro-cartilaginous structure (commonly referred to as an intervertebral disc) 13, while the second and third vertebrae 11 and 12 are separated by a second intervertebral disc 14. In the illustrated embodiment, the first intervertebral disc 13 is undamaged, but the second intervertebral disc 14 has been damaged, such as might occur as a result of an injury or degenerative causes. FIG. 2 shows the three vertebrae 10, 11, and 12 after a portion of the damaged intervertebral disc 14 has been removed. Typically, the nucleus pulposus (which is a jelly-like substance in the middle of the interior portion of the damaged intervertebral disc 14) is removed to allow a bone graft or other implantable material 100 to be deposited therein. FIG. 2 also shows a portion of an integrated device, indicated generally at 20, for the storage and delivery of the bone graft material 100 to the area formerly occupied by the nucleus pulposus. Although this invention will be described and illustrated in the context of the intervertebral damaged disc 14 and the fusion of the adjacent vertebrae 11 and 12, it will be appreciated that this invention may be practiced in connection with any other desired surgical procedure and for any other desired purpose. For example, this invention may also be used for other surgical procedures, such as posterolateral spinal fusion and revision hip arthroplasty.

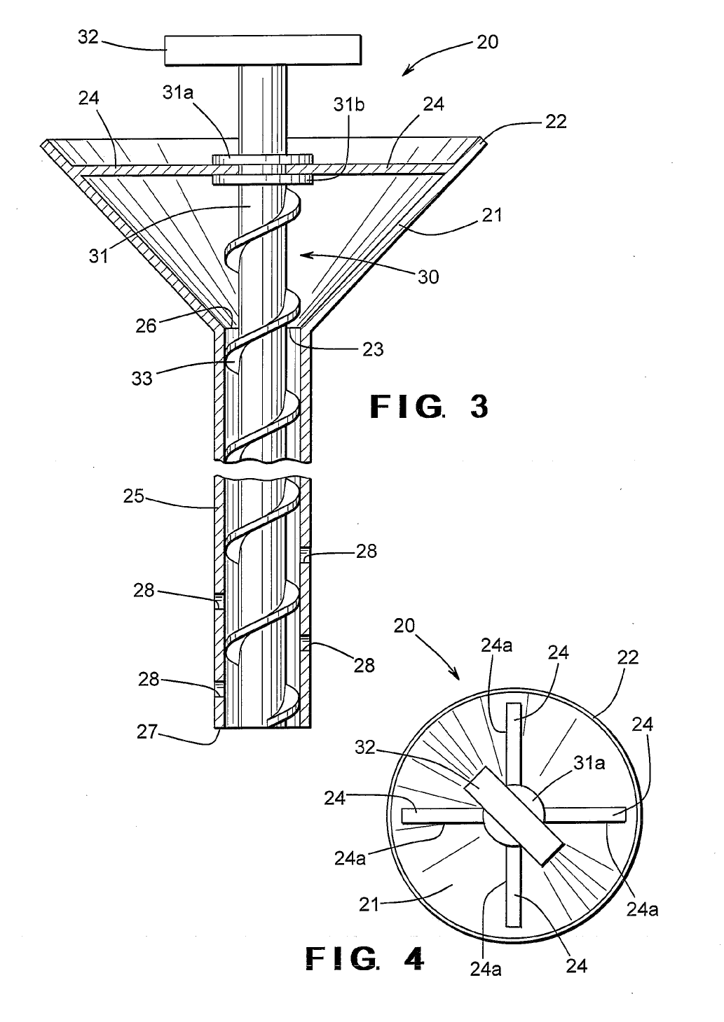

FIGS. 3 and 4 show a first embodiment of the integrated storage and delivery device 20 illustrated in FIG. 2. As shown therein, the integrated storage and delivery device 20 includes a storage portion 21. The illustrated storage portion 21 is hollow and frusto-conical in shape, tapering from a relatively large sized first open end 22 to a relatively small sized second open end 23. The illustrated storage portion 21 is symmetrical in shape and, therefore, defines a central axis therethrough. However, the storage portion 21 of the integrated storage and delivery device 20 may be formed having any desired shape.

In the illustrated embodiment, a closure member 24 is provided on the storage portion 21 near the first end thereof. The illustrated closure member 24 is a plurality (four in the illustrated embodiment) of support arms that are formed integrally with the storage portion 21. However, any number of such support arms 24 may be provided, and such support arms 24 may, if desired, be formed separately from the storage portion 21. The illustrated support arms 24 are spaced apart from the relatively large sized first end 22, are equidistantly spaced about the storage portion 21, and extend radially inwardly from the storage portion 21 toward the central axis. However, the support arms 24 may be provided at any desired location on the storage portion 21. Between adjacent ones of the storage arms 24, openings 24a are defined. In the illustrated embodiment, the storage arms 24 are relatively small in circumferential extent. Thus, the openings 24a are relatively large in circumferential extent. However, the storage arms 24 and the openings 24a may be formed having any desired circumferential sizes and shapes.

The integrated storage and delivery device 20 also includes a delivery portion 25. The illustrated delivery portion 25 is hollow and cylindrical in shape, extending from a first open end 26 to a second open end 27. However, the delivery portion 25 of the integrated storage and delivery device 20 may be formed having any desired shape. In the illustrated embodiment, the first end of the delivery portion 25 is formed integrally with the relatively small sized second end 23 of the storage portion 21. However, such is not required, as will become apparent below. The illustrated delivery portion 25 is symmetrical in shape and, therefore, defines a central axis therethrough that, in the illustrated embodiment, is co-axial with the central axis defined by the storage portion 21, although again such is not required. If desired, one or more lateral delivery openings 28 may be provided in the delivery portion 25 of the integrated storage and delivery device 20. The lateral delivery openings 28 may be provided at any desired locations on the delivery portion 25, but preferably are provided near the second open end 27 thereof. The purpose for such lateral delivery openings 28 will be explained below.

The integrated storage and delivery device 20 further includes a delivery mechanism, indicated generally at 30, for moving the bone graft material 100 from the storage portion 21 through the delivery portion 25 to a desired surgical site. In the illustrated embodiment, the delivery mechanism 30 is an auger that includes an auger shaft 31 that extends throughout the storage portion 21 and the delivery portion 25 of the integrated storage and delivery device 20. The illustrated auger shaft 31 is generally cylindrical in shape, although any desired shape is contemplated within the scope of this invention.

The auger shaft 31 is supported on the integrated storage and delivery device 20 for movement relative thereto. In the illustrated embodiment, the auger shaft 31 is supported on the integrated storage and delivery device 20 for rotational movement relative thereto by first and second support members 31a and 31b that are formed integrally with or otherwise provided on the auger shaft 31. The first and second support members 31a and 31b are sized, shaped, and positioned as desired to receive one or more of the support arms 24 therebetween, as shown in FIG. 3. As a result, the auger shaft 31 is supported on such support arms 24 for rotational movement relative to the storage portion 21 of the integrated storage and delivery device 20. A handle 32 is provided at an end of the auger shaft 31 adjacent to the first relatively large end of the storage portion 21. The handle 32 may be formed integrally with or separately from the auger shaft 31 as desired and may have any desired shape or combination of shapes that facilitate the convenient grasping and rotation thereby by a user of the integrated storage and delivery device 20.

The auger shaft 31 has a helical blade 33 or other material driving structure provided thereon. In the illustrated embodiment, the helical blade 33 is provided on the outer surface of the auger shaft 31 defines a constant outer diameter that extends throughout most of the length thereof, although such is not required. The outer diameter defined by the illustrated helical blade 33 is slightly smaller than an inner diameter defined by the delivery portion 25 of the integrated storage and delivery device 20. Thus, as will be described in greater detail below, when the auger shaft 31 is rotated relative to the integrated storage and delivery device 20, bone graft material 100 disposed within the storage portion 21 will be advanced through the delivery portion 25 to a surgical site.

The operation of the integrated storage and delivery device 20 will now be described. Initially, a quantity of the bone graft material 100 is disposed within the storage portion 21. To accomplish this, the bone graft material 100 may be poured or otherwise transferred from a storage container (not shown) supplied from a manufacturer into the relatively large sized first open end 22 of the storage portion 21 of the integrated storage and delivery device 20. This can be accomplished quickly and easily, without undesirable splashing or spilling, because of the relatively large size of the first open end 22 and the relatively large circumferential extents of the openings 24a defined between the support arms 24.

The storage portion 21 of the integrated storage and delivery device 20 can alternatively be pre-filled with the bone graft material 100 prior to the beginning of the surgical procedure so that it is ready to use when needed. Alternatively, the storage portion 21 of the integrated storage and delivery device 20 can be pre-filled with the bone graft material 100 from the manufacturer and sold therewith as a ready-to-use unit. This would eliminate the initial step of pouring or otherwise transferring the bone graft material 100 as described above.

Although not shown, the storage portion 21 of the integrated storage and delivery device 20 can be provided with one or more relatively small drainage openings. The bone graft material 100 is typically provided from the manufacturer in combination with a conventional cryopreservative material. Before such bone graft material 100 is used, it is desirable to remove this cryopreservative material and increase the temperature of the bone graft material 100. This is typically accomplished by diluting the mixture of the bone graft material 100 and the cryopreservative material with a conventional saline solution. The small drainage openings in the storage portion 21 of the integrated storage and delivery device 20 function as a sieve, allowing the saline solution and the cryopreservative material to drain from the storage portion 21, but retaining the bone graft material 100 therein for subsequent use.

When the surgical site has been adequately prepared, the integrated storage and delivery device 20 can be used to introduce the bone graft material 100 therein. For example, as shown in FIG. 2, after the portion of the intervertebral disc 14 has been removed, the integrated storage and delivery device 20 is positioned such that the delivery portion 25 (and, in particular, the second open end 27 and the lateral delivery openings 28 thereof) are located within the intervertebral disc 14 between the vertebrae 11 and 12, where it is desired to provide the bone graft material 100 to fuse such vertebrae 11 and 12 together.

Next, the delivery mechanism 30 of the integrated storage and delivery device 20 is actuated to move the bone graft material 100 from the storage portion 21 through the delivery portion 25 to the surgical site. In the illustrated embodiment, this can be accomplished by grasping the handle 33 and rotating the auger shaft 31 relative to the remainder of the integrated storage and delivery device 20. As a result, the rotating auger blade 32 draws the bone graft material 100 from the storage portion 21 and pushes it through the delivery portion 25. Some of the bone graft material 100 will be extruded laterally outwardly from the delivery portion 25 through the lateral delivery openings 28 thereof, while other portions of the bone graft material 100 will be forced longitudinally outwardly from the delivery portion 25 through the second open end 27 thereof. If desired, either of the lateral delivery openings 28 and the second open end of the deliver portion 25 may be closed to more precisely control the flow of the bone graft material 100 to the surgical site.

As mentioned above, the integrated storage and delivery device 20 of this invention eliminates the initial step of pouring or otherwise transferring the bone graft material 100 as described above, which can be problematic. Additionally, the delivery mechanism 30 of this invention is well suited for delivering a metered or otherwise precise amount of the bone graft material 100 at the surgical site in a well controlled manner. For example, the delivery mechanism 30 can be calibrated such that a predetermined amount of the bone graft material 100 is delivered at the surgical site for each partial or complete rotation of the handle 32. In this manner, the amount of the bone graft material 100 delivered at the surgical site can be quickly and easily controlled.

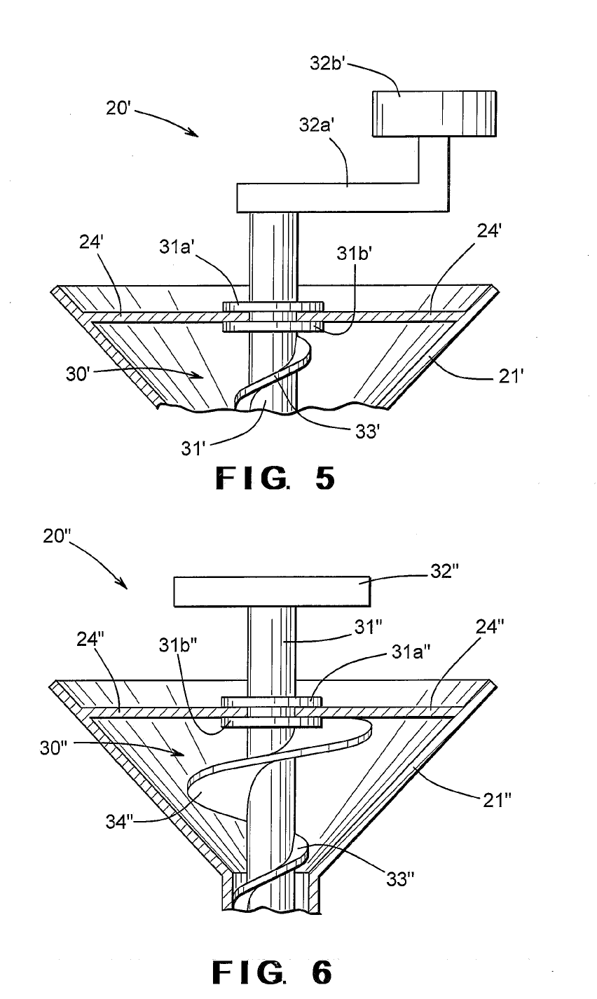

FIG. 5 illustrates a portion of a second embodiment of an integrated storage and delivery device, indicated generally at 20′, in accordance with this invention. The second embodiment of the integrated storage and delivery device 20′ is similar to the first embodiment 20 described above, and like reference numbers are used to indicate similar structures. In the second embodiment of the integrated storage and delivery device 20′, however, a modified handle is provided. The modified handle includes a laterally extending crank arm 32a′ having a knob 32b′ rotatably supported thereon. The crank arm 32a′ may be formed integrally with or separately from the auger shaft 31 as desired. The modified handle is otherwise identical in structure and operation to the above-described handle 32.

FIG. 6 illustrates a portion of a third embodiment of an integrated storage and delivery device, indicated generally at 20″, in accordance with this invention. The third embodiment of the integrated storage and delivery device 20″ is similar to the first embodiment 20 described above, and like reference numbers are used to indicate similar structures. In the third embodiment of the integrated storage and delivery device 20″, however, the auger blade 33″ includes an enlarged region 34″ that is disposed within the storage portion 21″. The enlarged region 34″ of the auger blade 33″ is provided to insure that all of the bone graft material 100 in the storage portion 21″ is drawn into the delivery portion 25″ and, therefore, delivered at the surgical site. The auger blade 33″ is otherwise identical in structure and operation to the above-described auger blade 33.

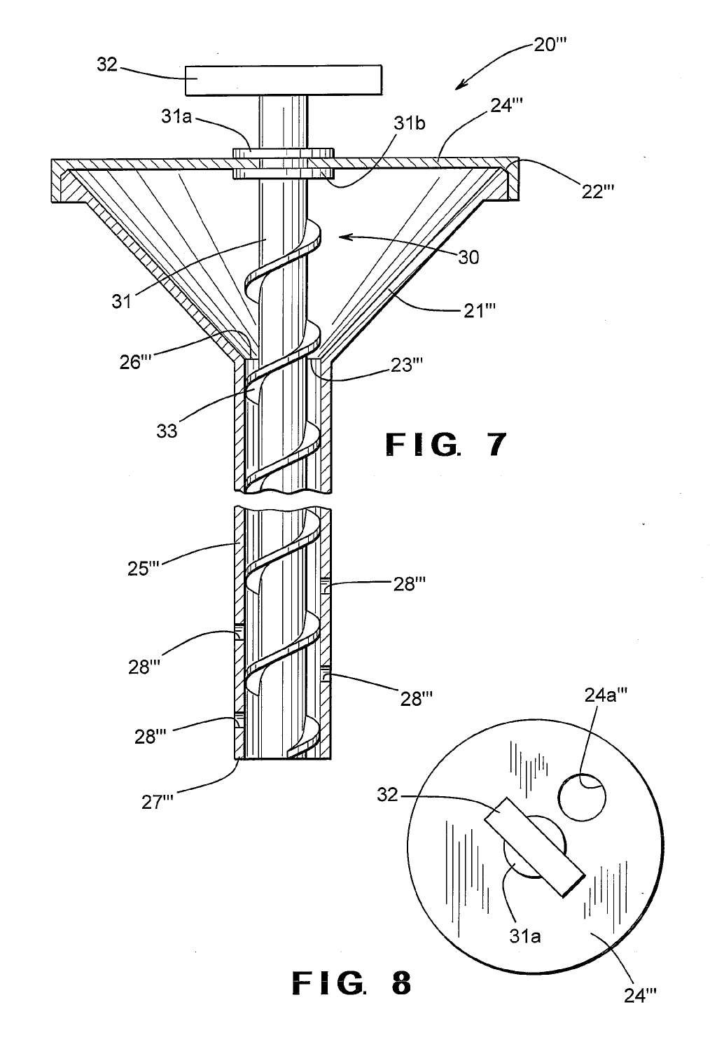

FIGS. 7 and 8 illustrate a portion of a fourth embodiment of an integrated storage and delivery device, indicated generally at 20′″, in accordance with this invention. The fourth embodiment of the integrated storage and delivery device 20′″ is similar to the first embodiment 20 described above, and like reference numbers are used to indicate similar structures. In the fourth embodiment of the integrated storage and delivery device 20′″, however, a separate and removable closure member 24′″ is provided in lieu of the closure member 24 described above. The illustrated removable closure member 24″′ is embodied as a cap having a circular end wall that extends across the relatively large sized first open end 22′″ of the delivery portion 21′″ and an annular side wall that extends circumferentially about the relatively large sized first open end 22′″. The auger shaft 31 is supported on the removable closure member 24′″ by the first and second support members 31a and 31b, which receive removable closure member 24′″ therebetween in a manner that is similar to that described above.

The removable closure member 24′″ can be releasably secured to the delivery portion 21′″ of the integrated storage and delivery device 20′″ in any desired manner including, for example, by frictional engagement, a positive snap-fit structure, a mutually threaded arrangement, and the like. If desired, an opening 24a′″ may be formed through the removable closure member 24′″ to permit the bone graft material 100 to be poured or otherwise transferred into the delivery portion 21′″ or to permit a scoop or similar tool (not shown) to be inserted therethrough to move such bone graft material 100 into engagement with the auger blade 33. The removable closure member 24′″ is otherwise identical in structure and operation to the above-described closure member 24.

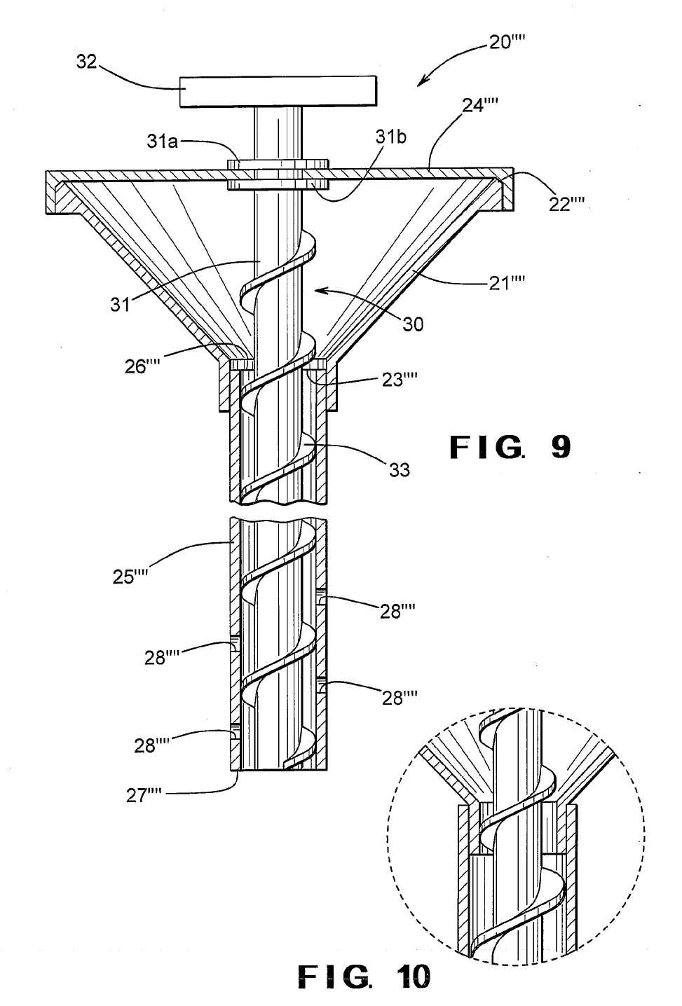

FIGS. 9 and 10 illustrate portions of a fifth embodiment of an integrated storage and delivery device, indicated generally at 20″″, in accordance with this invention. The fifth embodiment of the integrated storage and delivery device 20″″ is similar to the fourth embodiment 20′″ described above, and like reference numbers are used to indicate similar structures. In the fifth embodiment of the integrated storage and delivery device 20″″, however, the storage portion 21″″ and the delivery portion 25″ are formed as separate pieces. This allows the bone graft material 100 to be supplied from a manufacturer in just the storage portion 21″″ of the integrated storage and delivery device 20″″. Typically, a storage cap (not shown) will be provided over the relatively small sized second open end 23″″ until the bone graft material 100 is needed for a surgical procedure. When that need arises, the storage cap is removed, and the storage portion 21″″ is connected to the delivery portion 25″″ for use. The storage portion 21″″ can be connected to the delivery portion 25″″ in any desired manner including, for example, by frictional engagement, a positive snap-fit structure, a mutually threaded arrangement, and the like. The storage portion 21″″ and the delivery portion 25″″ are otherwise identical in structure and operation to the above-described storage portion 21 and delivery portion 25. In FIG. 9, the relatively small sized second open end 23″″ is disposed about the first open end 26″″ of the delivery portion 25″″. In FIG. 10, the relatively small sized second open end 23″″ is disposed within the first open end 26″″ of the delivery portion 25″″.

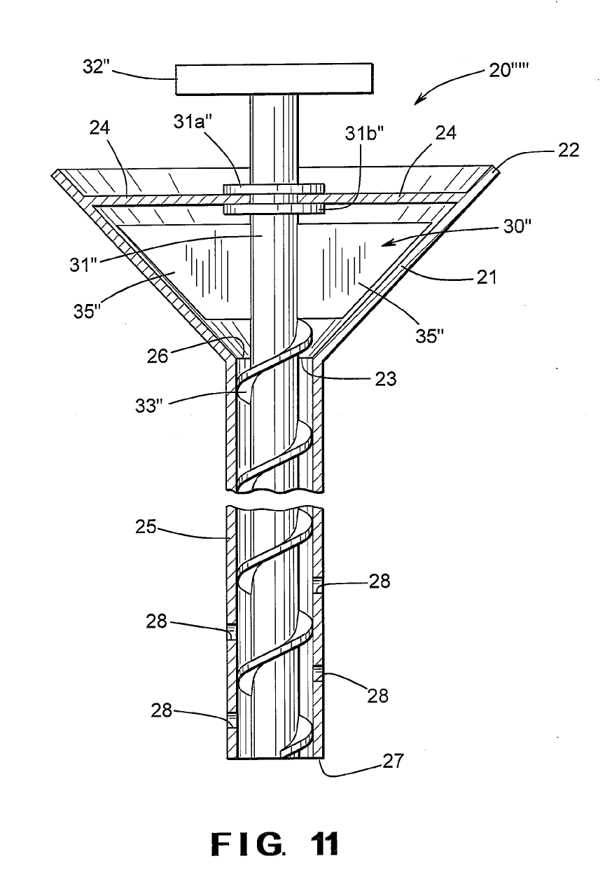

FIG. 11 illustrates a portion of a sixth embodiment of an integrated storage and delivery device, indicated generally at 20′″″, in accordance with this invention. The sixth embodiment of the integrated storage and delivery device 20′″″ is similar to the first embodiment 20 described above, and like reference numbers are used to indicate similar structures. In the sixth embodiment of the integrated storage and delivery device 20′″″however, the auger blade 33′″″ includes one or more paddles 35′″″ disposed within the storage portion 21. In the illustrated embodiment, two flat paddles 35′″″ extend laterally in opposite directions from one another. However, any desired number of paddles 35′″″ having any desired shape or combination of shapes may be provided. The paddles 35′″″ are provided to insure that all of the bone graft material 100 in the storage portion 21″ is urged into the delivery portion 25″ and, therefore, delivered at the surgical site. The paddles 35′″″ may be straight, spiral, or any other configuration to facilitate movement of the bone graft material 100 distally toward the delivery portion 25. The auger blade 33′″″ is otherwise identical in structure and operation to the above-described auger blade 33.

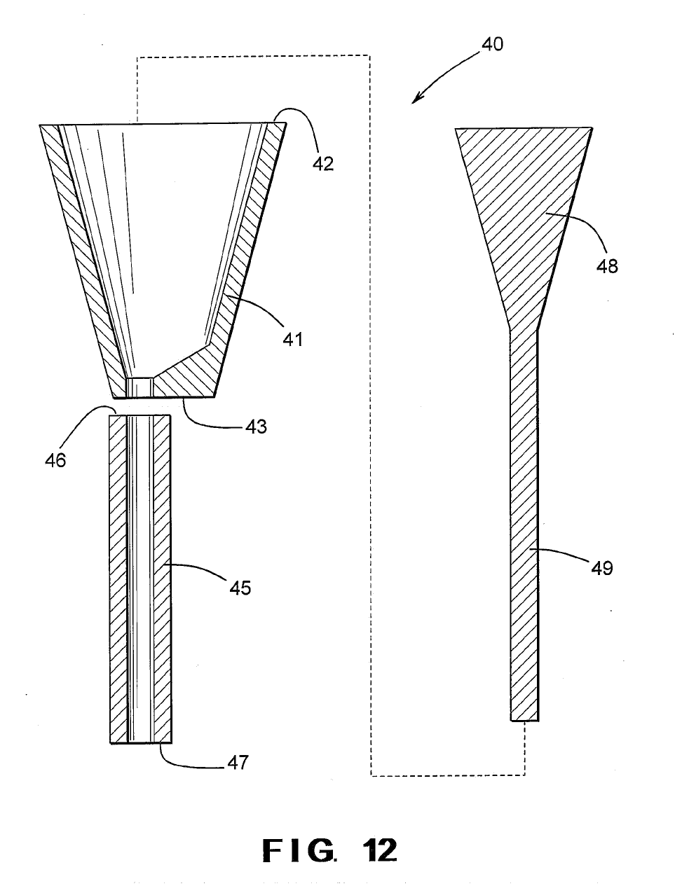

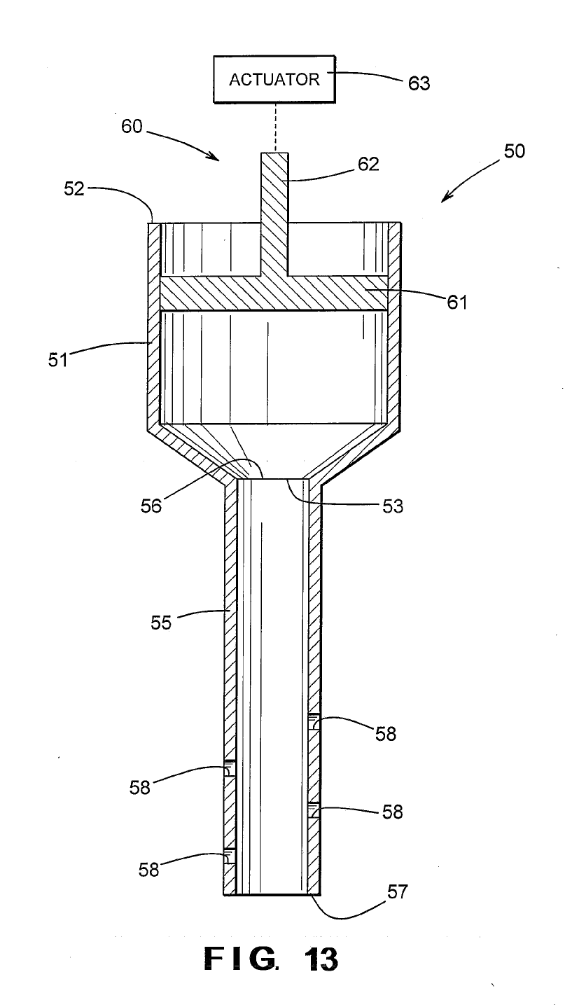

FIG. 12 illustrates a seventh embodiment of an integrated storage and delivery device, indicated generally at 40, in accordance with this invention. The integrated storage and delivery device 40 includes a storage portion 41 that, in the illustrated embodiment, is hollow and frusto-conical in shape, tapering from a relatively large sized first open end 42 to a relatively small sized second open end 43. However, the storage portion 41 of the integrated storage and delivery device 40 may be formed having any desired shape. The storage portion 41 defines a central axis therethrough.

The integrated storage and delivery device 40 also includes a delivery portion 45. The illustrated delivery portion 45 is hollow and cylindrical in shape, extending from a first open end 46 to a second open end 47. However, the delivery portion 45 of the integrated storage and delivery device 40 may be formed having any desired shape. In the illustrated embodiment, the first end of the delivery portion 45 is formed separately from the relatively small sized second end 43 of the storage portion 41. However, the first end of the delivery portion 45 may be formed integrally with the relatively small sized second end 43 of the storage portion 41 if desired. The delivery portion 45 defines a central axis therethrough that, in the illustrated embodiment, is axially offset from the central axis defined by the storage portion 41, although again such is not required.

The integrated storage and delivery device 40 further includes a delivery mechanism for moving the bone graft material 100 from the storage portion 41 through the delivery portion 45 to a desired surgical site. In the illustrated embodiment, the delivery mechanism is a manual plunger that includes an handle portion 48 and a shaft portion 49. The illustrated handle portion 48 is generally frusto-conical in shape, but the handle portion 48 may be formed having any desired shape that facilitates the grasping thereof by a user. The illustrated shaft portion 49 is generally cylindrical in shape, although any desired shape is contemplated within the scope of this invention. Preferably, the shaft portion 49 of the plunger defines an outer diameter that is slightly smaller than an inner diameter defined by the delivery portion 45 of the integrated storage and delivery device 40.

The storage portion 41 and the delivery portion 45 are preferably formed as separate pieces. This allows the bone graft material 100 to be supplied from a manufacturer in just the storage portion 41 of the integrated storage and delivery device 40. Typically, storage caps (not shown) will be provided over both the relatively large open end 42 and the relatively small sized second open end 43 until the bone graft material 100 is needed for a surgical procedure. When that need arises, the storage caps are removed, and the storage portion 41 is connected to the delivery portion 45 for use. The storage portion 41 can be connected to the delivery portion 45 in any desired manner including, for example, by frictional engagement, a positive snap-fit structure, a mutually threaded arrangement, and the like. Thereafter, the shaft portion 49 of the plunger can be inserted through the storage portion 41 and into the delivery portion 45 to manually move the bone graft material 100 to a desired surgical site.

FIG. 11 illustrates an eighth embodiment of an integrated storage and delivery device, indicated generally at 50, in accordance with this invention. The integrated storage and delivery device 50 includes a storage portion 51 that, in the illustrated embodiment, is hollow and frusto-conical in shape, tapering from a relatively large sized first open end 52 to a relatively small sized second open end 53. However, the storage portion 51 of the integrated storage and delivery device 50 may be formed having any desired shape. The storage portion 51 defines a central axis therethrough.

The integrated storage and delivery device 50 also includes a delivery portion 55. The illustrated delivery portion 55 is hollow and cylindrical in shape, extending from a first open end 56 to a second open end 57. However, the delivery portion 55 of the integrated storage and delivery device 50 may be formed having any desired shape. In the illustrated embodiment, the first end of the delivery portion 55 is formed integrally with the relatively small sized second end 53 of the storage portion 51. However, the first end of the delivery portion 55 may be formed separately from the relatively small sized second end 53 of the storage portion 51 if desired. The delivery portion 55 defines a central axis therethrough that, in the illustrated embodiment, is co-axial with the central axis defined by the storage portion 51, although again such is not required. If desired, one or more lateral delivery openings 58 may be provided in the delivery portion 55 of the integrated storage and delivery device 50. The lateral delivery openings 58 may be provided at any desired locations on the delivery portion 55, but preferably are provided near the second end 57 thereof.

The integrated storage and delivery device 50 further includes a delivery mechanism, indicated generally at 60, for moving the bone graft material 100 from the storage portion 51 through the delivery portion 55 to a desired surgical site. In the illustrated embodiment, the delivery mechanism 60 includes a piston or similar plunger mechanism 61 that is disposed within the storage portion 51 of the integrated storage and delivery device 50 for sliding movement relative thereto. The piston 61 is connected through a rod 62 to an actuator 63. The actuator 63 can be embodied as any device that can selectively effect sliding movement of the piston 61 relative to the storage portion 51 of the integrated storage and delivery device 50. For example, the actuator 63 can be embodied as a conventional mechanical, electrical, hydraulic, or pneumatic actuator that, when actuated, causes the piston 61 to move relative to the storage portion 51 of the integrated storage and delivery device 50. When the piston 61 is caused to move from the relatively large sized first open end 52 of the storage portion 51 toward the relatively small sized second open end 53, the bone graft material 100 contained within the storage portion 51 is moved through the delivery portion 55 to a desired surgical site. When the piston 61 is caused to move from the relatively small sized second open end 53 of the storage portion 51 toward the relatively large sized first open end 52, additional bone graft material 100 can be disposed within the storage portion 51 for future use.

The above detailed description of this invention is given for explanatory purposes. It will be apparent to those skilled in the art that numerous changes and modifications other than those cited can be made without departing from the scope of the invention. Accordingly, the whole of the foregoing description is to be construed in an illustrative and not a limitative sense, the scope of the invention being defined by the appended claims.

What is claimed is:

1. A device for the storage and delivery of a bone graft or other implantable material to a desired surgical site comprising:
a storage portion that extends from a first end to a second end, the first end of the storage portion adapted to have a bone graft or other implantable material transferred therethrough into the storage portion;
a delivery portion that extends from a first end to a second end, the first end of the delivery portion communicating with the second end of the storage portion to permit the movement of the bone graft or other implantable material therethrough, the second end of the delivery portion adapted to be positioned at a desired surgical site; and
a delivery mechanism that is provided within the storage portion and the delivery portion and is adapted to move the bone graft material from the storage portion through the delivery portion to a desired surgical site.

2. The device defined in claim 1 wherein the first end of the delivery portion is formed integrally with the second end of the storage portion.

3. The device defined in claim 1 wherein the first end of the delivery portion is formed separately from the second end of the storage portion.

4. The device defined in claim 3 wherein the first end of the delivery portion is releasably connected to the second end of the storage portion.

5. The device defined in claim 1 further including a closure member provided on the storage portion adjacent the first end thereof.

6. The device defined in claim 5 wherein the closure member is formed integrally with the storage portion.

7. The device defined in claim 5 wherein the closure member is formed separately from the storage portion.

8. The device defined in claim 5 wherein the delivery device is supported on the closure member.

9. The device defined in claim 1 wherein the delivery portion includes one or more lateral delivery openings adjacent to the second end thereof.

10. The device defined in claim 1 wherein the storage portion is symmetrical in shape and defines a central axis therethrough, and wherein the delivery portion is symmetrical in shape and defines a central axis therethrough that is co-axial with the central axis defined by the storage portion.

11. The device defined in claim 1 wherein the storage portion is symmetrical in shape and defines a central axis therethrough, and wherein the delivery portion is symmetrical in shape and defines a central axis therethrough that is offset from the central axis defined by the storage portion.

12. The device defined in claim 1 wherein the delivery mechanism is movable relative to the storage portion and the delivery portion to move the bone graft material from the storage portion through the delivery portion to a desired surgical site.

13. The device defined in claim 12 wherein the delivery mechanism is rotatable relative to the storage portion and the delivery portion to move the bone graft material from the storage portion through the delivery portion to a desired surgical site.

14. The device defined in claim 13 wherein the delivery mechanism includes a handle to facilitate rotation of the delivery mechanism relative to the storage portion and the delivery portion.

15. The device defined in claim 13 wherein the delivery mechanism includes an auger blade.

16. The device defined in claim 15 wherein the auger blade includes a first portion that is disposed within the storage portion and a second portion that is disposed within the delivery portion, and wherein the first and second portions of the auger blade have different sizes.

17. The device defined in claim 15 wherein the delivery mechanism further includes one or more paddles that are disposed within the storage portion, and wherein the auger blade is disposed within the delivery portion.

18. The device defined in claim 1 wherein the delivery mechanism is a plunger mechanism that is axially movable relative to the storage portion and the delivery portion to move the bone graft material from the storage portion through the delivery portion to a desired surgical site.

19. The device defined in claim 18 further including an actuator for axially moving the delivery mechanism relative to the storage portion and the delivery portion.

20. A device for the storage of a bone graft or other implantable material and for the delivery of the bone graft or other implantable material to a desired surgical site.
